(19) United States
(12) Patent Application Publication
(10) Pub. No.: US 2013/0345596 A1
(43) Pub. Date: Dec. 26, 2013

(54) APPARATUS AND METHODS FOR REMOVING AND COLLECTING BIOPSY SPECIMENS FROM BIOPSY DEVICES WITH FIXATION AND PREPARATION FOR HISTOPATHOLOGICAL PROCESSING OR OTHER ANALYSIS

(71) Applicant: David S. ZIMMON, Port Washington, NY (US)

(72) Inventor: David S. ZIMMON, Port Washington, NY (US)

(21) Appl. No.: 13/923,678

(22) Filed: Jun. 21, 2013

Related U.S. Application Data
(60) Provisional application No. 61/690,389, filed on Jun. 25, 2012.

(51) Int. Cl. A61B 10/02

(52) U.S. Cl. CPC A61B 10/02
USPC 600/562

(57) ABSTRACT

An apparatus and method for removing biopsy specimens from spring based spring based multiple biopsy or forceps biopsy devices by irrigating or suctioning. The apparatus collects, fixes and prepares specimens for histopathologic processing limiting manual labor, workplace risk and time previously required. After removal from the biopsy device, specimens are fixed in a histopathological processing cassette or on specimen wrapping paper for transportation to the pathology laboratory. In pathology, fixative is drained and specimens inspected for gross description followed by paper wrapping and histopathological processing with minimum handling, exposure to infectious material or toxic fixatives. After removing the biopsy specimens, the biopsy device is washed in the apparatus to eliminate contaminants and removed, ready for another biopsy sequence. A spring based spring based multiple biopsy device using these methods can collect numerous biopsy batches from different sites with segregated processing and analysis of each batch.

### CROSS-REFERENCE TO RELATED APPLICATIONS

This application claims priority under 35 USC 119(e) of U.S. Provisional Application Ser. No. 61/690,389, filed on Jun. 25, 2012.

### BACKGROUND OF THE INVENTION

1. Field of the Invention

The present invention relates to removing and collecting biopsy specimens from biopsy devices with fixing for analysis by histopathological, biological or physical methods. Biopsy specimens are minute pieces of fragile tissue that are difficult to remove from the biopsy device used to cut or scrape them from tissue. Biopsy specimens require careful handling to prevent loss, fragmentation and contamination. After manual removing from the biopsy instrument specimens require fixing and collecting for analysis. Fixed specimens must be removed from fixative, inspected and prepared for histopathological processing. This laborious process is slow and costly.

Spring based spring based multiple biopsy devices take many biopsies in a single pass to the target tissue. Specimens are collect serially in acquisition order within a metal or plastic storage cylinder in the biopsy instrument tip for in situ fixation and histopathological processing. In situ fixation and processing within the storage cylinder requires separating the spring based multiple biopsy storage cylinder from the shaft, thereby destroying the spring based multiple biopsy device. A method is needed for collecting spring based multiple biopsy specimens individually or as batches for analysis but still retaining the advantages of acquiring many specimens in each pass to the tissue. Further forceps biopsy and other biopsy instruments that collect single or a few specimens need improved methods for removing, collecting and processing specimens.

2. The Prior Art

Many biopsy devices collect only 1 to 2, or at most 4 specimens with each tissue pass. Numerous biopsy passes through an access path, operative instrument or endoscope are required to collect adequate quantities of tissue and to segregate each biopsy site for identification. Specimens from each biopsy pass are retrieved through the access path by withdrawing the biopsy device from the biopsy site, manually removing the specimen(s) from the biopsy device and placing the specimen(s) in a labeled container to identify the biopsy site, indication and patient. Minute specimens may be damaged or lost during this process. Specimens are contaminated during retrieval and lose orientation and biopsy sequence in the container. During these operations staff is exposed to sharps, infectious material and toxic fixatives. After each biopsy pass and specimen removal the biopsy device is washed before the next biopsy pass. A second operator is needed to perform these tasks. These actions prolong the operation and anesthesia with increased patient risk and cost.

In the pathology laboratory, specimens obtained from each biopsy site are processed separately to maintain identification during histopathological processing, slide fabrication and diagnostic microscopic examination. A pathology laboratory receives numerous specimens from each patient in many fixative containers. Each container must be opened and inspected to record the number, size and source of the specimens. Then each container specimen batch is filtered on to a paper wrap to remove fixative and transferred to individual processing cassettes labeled for identification. During this process the minute specimens are subject to damage, fragmentation and loss. These manual operations expose staff to infectious material and toxic fixatives. Specimens for chemical, biological or genetic testing may require biopsies that are handled separately. This tedious, labor intensive process is expensive in staff required, time, equipment and laboratory space.

U.S. Pat. Nos. 5,685,320, 5,782,747, 6,071,248 and 7,445,603 to Zimmon, herein incorporated by reference, describe a spring based spring based multiple biopsy device that cuts and captures a series of biopsy specimens in a single pass to the biopsy site. Specimen are collected, oriented and stored in acquisition order inside a metal or plastic storage cylinder. The storage cylinder is separated from the biopsy device and placed in a fixative container. In the pathology laboratory, the fixed storage cylinders are processed in situ with the specimen inside. Spring based spring based multiple biopsy devices collect numerous specimens in a single pass to the tissue. But in situ fixation and processing destroys the biopsy device preventing additional passes for collecting specimens. Forceps biopsy devices use multiple passes to the tissue requiring manual removing of single or at most a few biopsies into fixative from each pass. In the pathology laboratory separate processing of each specimen batch is necessary. The prior biopsy art needs improving in removing specimens from the biopsy device, collecting, fixing and pathological processing of biopsy specimens.

### SUMMARY OF THE INVENTION

It is the object of the present invention to provide an apparatus and method for removing and collecting biopsy specimens individually or as batches from spring based multiple biopsy storage cylinders or other biopsy devices combined with fixation, fixative removal and paper wrapping or collecting in a cassette for histopathological processing or other analysis. The invention does not damage the spring based multiple biopsy device, allowing a single instrument to make numerous spring based multiple biopsy passes. The invention minimizes manual specimen handling and limits staff exposure to sharps, infectious material and toxic fixatives. After specimen removal, the biopsy device is washed in the specimen collection apparatus and when removed is available immediately for reuse in the same patient. Spring based multiple biopsy device utility is improved by allowing the collecting of numerous specimen batches from more than one biopsy pass in each patient. The invention prepares biopsy specimens for processing or other analysis by collecting specimens in a receptacle for fixing. The receptacle contains a removable perforated plastic screen supporting specimen wrapping paper or a histopathological processing cassette, a bottom drain for removing fixative and a lid. In the pathology laboratory draining fixative is followed by removing the plastic screen with wrapping paper or the processing cassette, inspecting the specimens and recording their size and number. Specimens on paper are wrapped for processing or the processing cassette is processed. The invention obviates the pathology laboratory prior manual opening of each fixative vial, retrieving the specimens, removing fixative by filtering on to wrapping paper, inspecting and paper wrapping fixed or unfixed specimens for processing.

The invention also includes a clear plastic single wall irrigating apparatus for removing and collecting biopsy specimens from spring based multiple biopsy storage cylinders or other biopsy devices.

In another embodiment, there is a clear plastic suctioning apparatus and method for removing and collecting biopsy specimens from spring based multiple biopsy storage cylinders or other biopsy devices in a removable receptacle for fixation, fixative removal and paper wrapping or collecting in a histopathological processing cassette or other analysis.

In another embodiment, there is a clear plastic double walled irrigation apparatus and method for removing and collecting biopsy specimens from spring based multiple biopsy storage cylinders or other biopsy devices in a removable receptacle for fixation, fixative removal and paper wrapping or collecting in a histopathological processing cassette or other analysis. The double cylinder embodiment is useful for narrow and or flexible biopsy devices where the inner cylinder must be narrow to facilitate passage of the biopsy instrument through the inner lumen or when toxic fixatives require a second cylinder wall to guard against environmental contamination.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a single walled clear plastic irrigating specimen removal apparatus for collecting spring based multiple biopsy specimens from perforated spring based multiple biopsy storage cylinders or other biopsy devices into a receptacle (not shown).

FIG. 2 shows the terminally ridged shaft or fissured distal cylinder seal of an open forceps biopsy device within the irrigating specimen removal apparatus for removing and collecting biopsy specimens in a receptacle (not shown).

FIG. 3 shows a suctioning specimen removal apparatus with a closed attached removable receptacle for collecting, fixing, draining fixative, inspecting and paper wrapping or collecting in a perforated histopathological processing cassette of spring based multiple biopsy device or forceps biopsy specimens with or without fixative in preparation for histopathological processing or other analysis.

FIG. 4 shows a double walled irrigating specimen removing apparatus for collecting, fixing, draining fixative, inspecting and paper wrapping or collecting in a perforated histopathological processing cassette of spring based multiple biopsy or forceps biopsy specimens in a closed attached removable receptacle with or without fixative in preparation for histopathological processing or other analysis;

FIG. 5 shows a receptacle 212 after specimen collection and separation from the specimen removal apparatus with a perforated histopathological processing cassette supported by the perforated removable plastic screen replacing specimen wrapping paper and a removable lid for closing the receptacle after specimen collection for transport to the pathology laboratory.

### DETAILED DESCRIPTION OF THE PREFERRED EMBODIMENTS

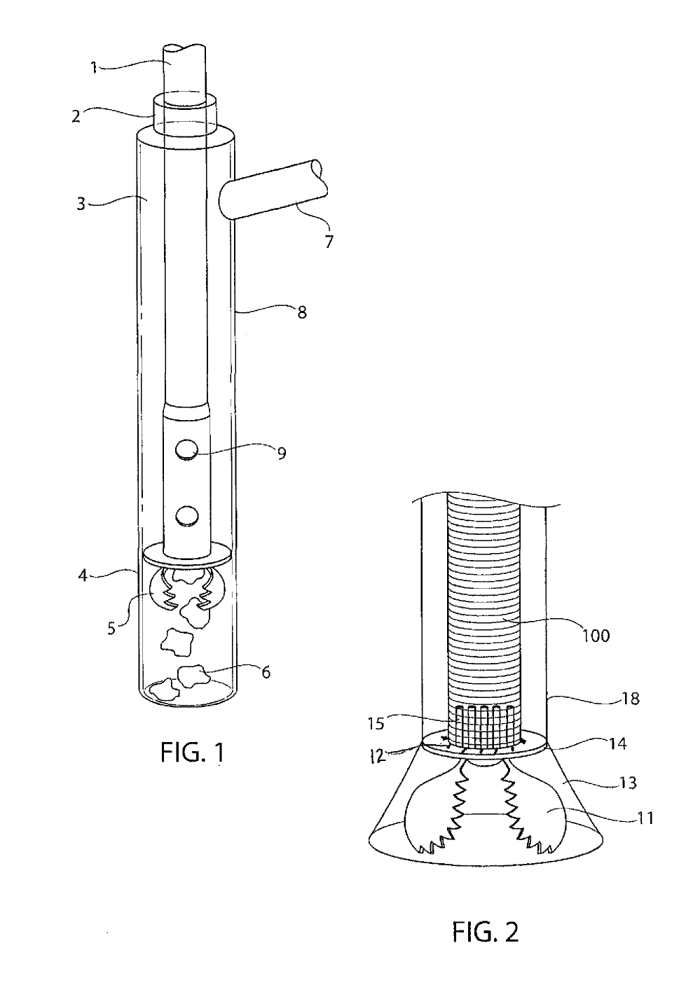

One embodiment of the invention as shown in FIG. 1 is a single wall clear plastic irrigating apparatus for collecting individual specimens or specimens groups from a spring based multiple biopsy storage cylinder or other biopsy devices. The closed biopsy device and shaft are passed through a proximal perforated elastic seal 2 down an irrigating apparatus lumen 3 and out the perforated distal seal 4. The spring based multiple biopsy device jaws are opened by activating the biopsy mechanism (not shown) to open an exit path for specimen 6 removal. Irrigating fluid or gas is injected through the side arm 7 into cylinder 8 of lumen 3.

Fluid passes into the lumen 3 through spring based multiple biopsy storage cylinder perforations 9 entraining the storage cylinder specimens 6 into a receptacle (not shown). After removing the specimens in the receptacle, the biopsy devices is washed by irrigating through the side arm 7 and removed ready for reuse.

FIG. 2 shows a forceps biopsy device having shaft 100 with terminal longitudinal ridges 15 or fissures 12 in the distal cylinder seal, and biopsy cups 11. The cups have a forceful jaw-fulcrum scissor action and are passed through lumen 18 with the biopsy cups 11 holding the specimen(s) closed. The biopsy cups 11 are pulled back and opened within the distal elastic seal 14 to spread the elastic seal 14, or the shaft 100 may be ridged longitudinally with ridges 15 or the distal cylinder seal may have fissures 12 to allow irrigating fluid to entrain the specimens into a receptacle (not shown). The terminal portion 13 of the apparatus beyond the distal seal 14 is flared to allow full opening of the forceps biopsy cups for specimen removal.

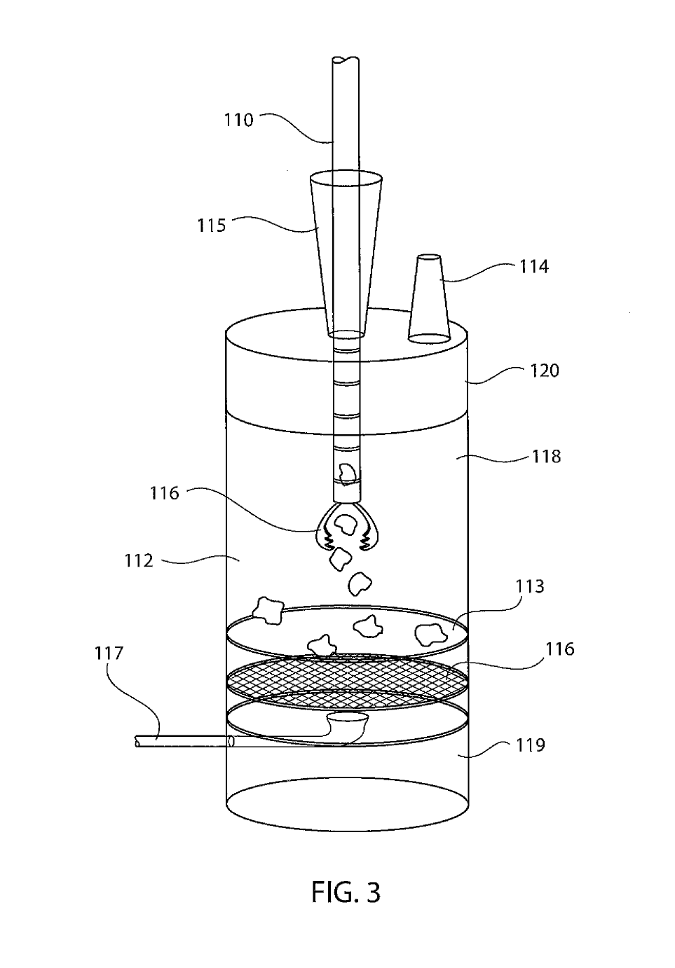

FIG. 3 shows another embodiment of the invention, having a clear plastic suctioning apparatus and method for collecting individual specimens or groups of specimens from spring based multiple biopsy storage cylinders or other biopsy devices by suctioning into a removable fixative receptacle 112 containing fixative and depositing the specimens on wrapping paper 113 or in a perforated plastic cassette (not shown) for histopathological processing.

A cylinder 118 of clear plastic has a port 114 for applying suction to the cylinder lumen. A narrow tube 115 sealed into the cylinder 118 extends above the cylinder 118 and into the lumen. The tube 115 is sized slightly larger than the biopsy device. The closed spring based multiple biopsy device or forceps biopsy containing specimens 16 is passed through the inlet tube into the cylinder lumen. The biopsy mechanism is activated to open the biopsy device and shaft 110 pulled back until the open biopsy device is below the inlet tube. Suctioning through the port 114 creates negative pressure in the cylinder 118, pulling air through the inlet tube 115. Entraining inlet air encourages the biopsy specimens to pass into fixative in the removable cylinder bottom 112. A screen (not shown) over the suction port 114 at the port entrance into the cylinder 118 prevents specimen loss from excess suction. The removable cylinder base supports a removable perforated plastic screen 116 supporting specimen wrapping paper 113 or a histopathological processing cassette (not shown) for collecting the specimens. A removable lid (not shown) is placeable over the removable lower cylinder after collecting specimens. A removable cylinder bottom 119 has a drain 117 for removing fixative before removing the cylinder lid 120 for inspecting and paper wrapping specimens or removing a histopathological processing cassette. After removing the cylinder base 119 and specimens, the biopsy device 110 is washed by irrigating through the inlet tube 115 and ready for reuse.

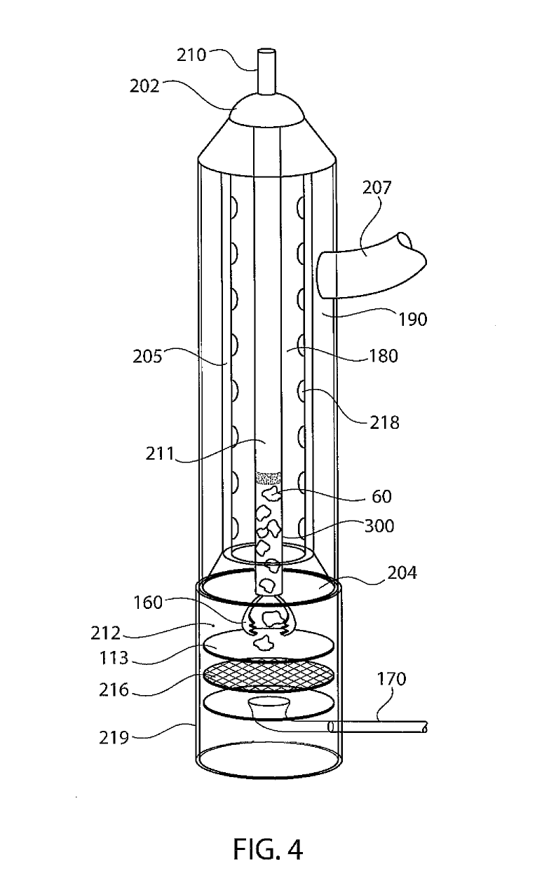

Another embodiment of the invention is a clear plastic double walled irrigating apparatus (FIG. 4) and method for collecting individual specimens or groups of specimens from spring based multiple biopsy storage cylinders or other biopsy devices by irrigating into a removable fixative receptacle formed by cylinder base 219, draining fixative via drain 170 and depositing specimens 160 on wrapping paper 113 or in a perforated histopathological cassette (not shown) for processing. The closed biopsy device 210 is placed in the inner cylinder 205 through the proximal elastic seal 202, down the inner cylinder lumen 180 and through the distal seal 204. The spring-based multiple biopsy device 210 is opened by activating the biopsy mechanism (not shown). The biopsy device 210 and shaft 211 are pulled back until the open biopsy device jaws 160 are below the inner cylinder and distal seal 204. Irrigating fluid or gas 212 is injected through the side arm 207 into the outer cylinder 190, passing through the inner cylinder perforations 218 into the inner cylinder lumen 180 and through spring based multiple biopsy storage cylinder perforations 300 to push specimens 60 out the open spring based multiple biopsy device open jaws exit path into the removable cylinder base 219.

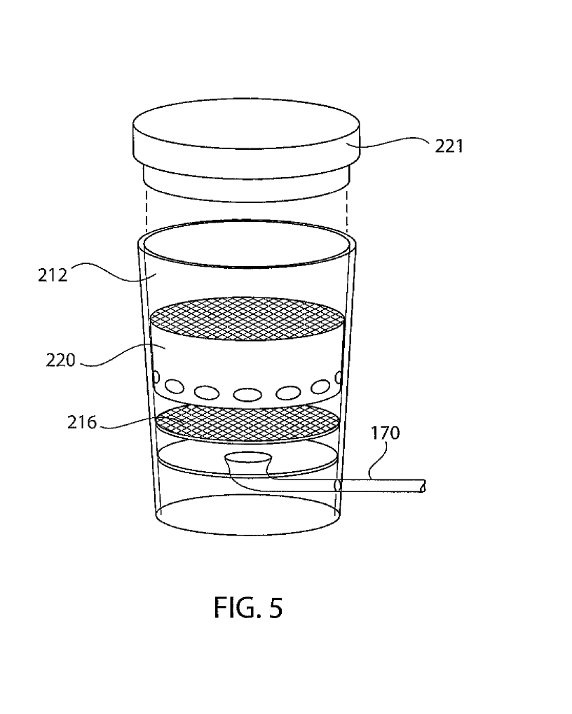

The removable cylinder base 219 supports a removable perforated plastic screen 216 supporting specimen wrapping paper 113 or a histopathological processing cassette 220, as shown in FIG. 5, for collecting the specimens. A removable lid 221 is placeable over the removable lower cylinder 219 after collecting specimens. The removable lower cylinder 219 has a drain 170 for removing fixative before removing the cylinder lid 221 for inspecting and paper wrapping specimens or removing a histopathological processing cassette. After removing the cylinder base 219 and specimens, the biopsy device 210 is washed by irrigating through the side arm 207 and is ready for reuse. Forceps biopsy devices with a forceful jaws 160 having a fulcrum scissor action (FIG. 2) are opened with the biopsy mechanism within the inner cylinder distal elastic seal 4 to spread the elastic seal orifice, or the distal shaft may by ridged longitudinally or the distal cylinder seal may be fissured (FIG. 2)to allow the pressured irrigation fluid to pass between the ridge grooves or fissured seal entraining the specimens into the removable lower cylinder.

Accordingly, while only a few embodiments of the present invention have been shown and described, it is obvious that many changes and modifications may be made thereunto without departing from the spirit and scope of the invention.

What is claimed is:

1. An apparatus for performing a medical procedure, comprising:
a single walled cylinder having proximal and distal ends,
a side arm connected to the cylinder for irrigating the cylinder with fluid or gas;
a forceps biopsy device having biopsy cups and a shaft and being disposed in said cylinder;
perforated elastic seals covering the proximal and distal ends, the seals having passage openings being sized to close around the shaft of the biopsy device;
a removable bottom section of the cylinder, said removable bottom section holding fixative; and
a drain disposed in the said removable bottom section for draining fixative.

2. The apparatus of claim 1, wherein said biopsy device is adapted to hold spring based multiple biopsy specimens.

3. The apparatus of claim 1, further comprising a perforated inner cylinder disposed in said cylinder and wherein said biopsy device passes through said perforated inner cylinder and through said distal seal to the cylinder bottom part.

4. The apparatus of claim 3, wherein extending said biopsy jaws and biopsy mechanism out of said cylinder causes irrigating fluid or gas injected through said cylinder side arm to enter the perforated spring based multiple biopsy housing and exit said spring based multiple biopsy open jaws to entrain said biopsy specimens into said cylinder bottom section.

5. The apparatus of claim 1, wherein the forceps biopsy device has a shaft with longitudinal ridging, such that irrigating fluid passing in grooves between said shaft ridging encourages said biopsy specimens into said removable cylinder bottom.

6. The apparatus of claim 1, wherein the distal cylinder seal has fissures, such that irrigating fluid passes through the fissures and encourages said biopsy specimens into said removable cylinder bottom.

7. The apparatus of claim 1, further comprising a removable perforated plastic screen disposed in said bottom section, said plastic screen supporting a removable wrapping paper or a histopathological processing cassette covering said removable plastic screen.

8. The apparatus of claim 1, further comprising a removable lid placeable over said removable cylinder bottom when separated from said cylinder.

9. The apparatus of claim 1, wherein said removable cylinder bottom is replaceable with a new bottom cylinder for removing, collecting and fixing said specimens from another biopsy device specimen collecting pass.

10. An apparatus for performing a medical procedure comprising:
a clear sealed plastic single walled cylinder with a port for applying suction to a lumen of the cylinder, said cylinder having a removable bottom section holding fixative,
a screen covering said suction port to prevent specimen loss into said suction source if excessive suction is applied; and
an inlet tube sealed into said suctioning cylinder extending above and into said cylinder lumen; and
a drain connected to said removable bottom section for removing said fixative.

11. The apparatus of claim 10, further comprising a biopsy device containing biopsy specimens passing through said inlet tube and extending into said cylinder lumen.

12. The apparatus of claim 11, wherein opening said biopsy device and pulling back to said inlet tube exposes said specimens to air drawn through said inlet tube by suctioning at said suction port.

13. The apparatus of claim 2, wherein suctioning at said suction port pulls air out of said cylinder, drawing air through said inlet tube entraining biopsy specimens from said biopsy device into said suctioning cylinder bottom containing said fixative.

14. The apparatus of claim 10, wherein said bottom section is covered by a removable perforated plastic screen supporting removable wrapping paper or a removable histopathological processing cassette.

15. The apparatus of claim 10, further comprising a lid placeable over said removable cylinder bottom when separated from said suctioning cylinder.
